(19) United States
(12) Patent Application Publication
(10) Pub. No.: US 2001/0024638 A1
(43) Pub. Date: Sep. 27, 2001

(54) Stable microbubble suspensions as enhancement agents for ultrasound echography and dry formulations thereof

(76) Inventors: Michel Schneider, Troinex (CH); Jean Brochot, Feigeres (FR); Jerome Puginier, Le Chable-Beaumont (FR); Feng Yan, Meyrin/Geneva (CH)

Correspondence Address: NIXON & VANDERHYE P.C., 1100 North Glebe Road, 8th Floor, Arlington, VA, 22201, US

(21) Appl. No.: 09/770,216

(22) Filed: Jan. 29, 2001

Related U.S. Application Data
(63) Continuation-in-part of application No. 09/151,651, filed on Sep. 11, 1998, now Pat. No. 6,187,288.
Application No. 09/151,651 is a division of application No. 08/863,592, filed on May 27, 1997, now Pat. No. 5,868,426.
Application No. 08/863,592 is a division of application No. 08/420,677, filed on Apr. 12, 1995, now Pat. No. 5,686,060.
Application No. 08/420,677 is a division of application No. 08/134,671, filed on Oct. 12, 1993, now Pat. No. 5,445,813.

(30) Foreign Application Priority Data
Nov. 2, 1992 (EP) 928108372

(51) Int. Cl. A61K 49/22

(52) U.S. Cl. 424/9.51; 424/9.52

(57) ABSTRACT

Disclosed are injectable suspensions of gas filled microbubbles in an aqueous carrier liquid usable as contrast agents in ultrasonic echography. The suspensions comprise amphipathic compounds of which at least one may be a laminarized phospholipid as a stabiliser of the microbubbles against collapse with time and pressure. The concentration of phospholipids in the carrier liquid is below 0.01% wt but is at least equal to or above that at which phospholipid molecules are present solely at the gas microbubble-liquid interface. Also disclosed are methods of preparation of the stable suspensions of gas filled microbubbles, as well as dry formulations which, upon reconstitution in an aqueous carrier liquid, will form the injectable suspensions of gas filled microbubbles of the invention and methods of making and using the same The preferred dry formulations are stable when stored over time and at temperatures above ambient temperature and further comprise a lipid acid preserving agent additive.

### RELATED APPLICATIONS

This application is a continuation-in-part of co-pending application U.S. Ser. No. 09/151,651, filed Sep. 11, 1998, which is a division of U.S. Ser. No. 08/863,592, filed Jun. 26, 1997, now U.S. Pat. No. 5,908,610, which is in turn a division of U.S. Ser. No. 08/420,677, filed Apr. 12, 1995, now U.S. Pat. No. 5,686,060, which is in turn a division of U.S. Ser. No. 07/134,671, filed Oct. 12, 1993, now U.S. Pat. No. 5,445,813, which in turn claims benefit of European Patent Application No. 92810837, filed Nov. 2, 1992. Each of these disclosures is hereby incorporated by reference in its entirety.

### TECHNICAL FIELD

The invention relates to injectable suspensions of gas filled microbubbles in an aqueous carrier comprising amphipathic compounds of which at least one is a phospholipid stabilizer of the microbubbles against collapse with time and pressure. The phospholipid stabilizer may be in a lamellar or laminar form. The invention also comprises a method of making stable suspensions of microbubbles usable as contrast agents in ultrasonic echography. The invention further comprises dry formulations which are stable when stored over time and at a range of temperatures and which may be reconstituted to yield the echogenic, injectable suspensions of gas filled microbubbles of the invention.

### BACKGROUND OF INVENTION

Use of suspensions of gas microbubbles in a carrier liquid as efficient ultrasound reflectors is well known in the art. The development of microbubble suspensions as echopharmaceuticals for enhancement of ultrasound imaging followed early observations that rapid intravenous injections can cause solubilized gases to come out of solution forming bubbles. Due to their substantial difference in acoustic impedance relative to blood, these intravascular gas bubbles are found to be excellent reflectors of ultrasound. Injecting into the blood-stream of living organisms suspensions of gas microbubbles in a carrier liquid strongly reinforces ultrasonic echography imaging, thus enhancing the visualisation of internal organs. Since imaging of organs and deep seated tissue can be crucial in establishing medical diagnosis a lot of effort is devoted to the development of stable suspensions of highly concentrated gas microbubbles which at the same time would be simple to prepare and administer, would contain a minimum of inactive species, would be capable of long storage and simple distribution. Many attempts towards a solution which will satisfy these criteria have been made, however, improvements would be desired.

It has been known from EP-A-0 077 752 (Schering) that suspensions of gas microbubbles can be made by mixing an aqueous solution of a surfactant with a solution of a viscosity enhancer as a stabilizer. The gas bubbles are introduced into the mixture by forcing the mixture of reagents and air through a small aperture. A suspension of CO₂ microbubbles may be obtained by addition of an acid to a mixture obtained from a solution containing a surfactant and sodium bicarbonate and a solution of the viscosity enhancer. Mixing the components however, is to be carried out just before use and the solution is to be consumed/injected immediately upon preparation. The disclosed surfactants (tensides) comprise lecithins; esters and ethers of fatty acids and fatty alcohols with polyoxyethylene and polyoxyethylated polyols like sorbitol, glycols and glycerol, cholesterol; and polyoxy-ethylene-polyoxypropylene polymers. Disclosed concentration of tensides in the suspension is between 0.01% and 10% wt and a preferred range is claimed to be between 0.5% to 5%. The viscosity enhancing and stabilizing compounds include for instance mono- and polysaccharides (glucose, lactose, sucrose, dextran, sorbitol); polyols, e.g. glycerol, polyglycols; and polypeptides like proteins, gelatin, oxypolygelatin, plasma protein and the like. The total amount of viscosity enhancing agent is limited to 0.5 and 50%. Use of polyoxypropylene-polyoxyethylene polymers (eg. Pluronic® F-68) as viscosity enhancing agent has also been disclosed. In the preferred example, equivalent volumes of tenside, a 0.5% by weight aqueous solution of Pluronic® F-68 (a polyoxypropylene-polyoxyethylene copolymer), and the viscosity enhancer (a 10% lactose solution) are vigorously shaken together under sterile conditions to provide a suspension of microbubbles. The suspension obtained lasted over 2 minutes and contained close to 50% of bubbles with a size below 50 μm. According to the document up to 50% of surfactants and/or viscosity enhancing agents may be employed, however, specific examples use between 1% and 4% of Pluronic® F-68.

An attempt toward a stable echogenic suspension is disclosed in WO-92/11873 (Beller et. al.). Aqueous preparations designed to absorb and stabilise microbubbles for use as an echographic contrasting agent are made with polyoxyethylene/polyoxypropylene polymers and negatively charged phospholipids such as phosphatidylglycerol, phosphatidylinositol, phosphatidylethanol-amine, phosphatidylserine as well as their lysoforms. The concentration range of phospholipids in the preparations may be between 0.01% and 5% by volume or weight, however, preparations with 1% of dipalmitoylphosphatidyl glycerol (DPPG) are specifically disclosed and claimed. In addition to the negatively charged phospholipids the compositions must contain between 0.1% and 10% of polymeric material (Pluronic® F-68). The total amount of solutes in the preparations is between 5.1% and 10.4%. The concentration of the microbubbles is not reported, however, according to the results given it may be estimated to be about 10⁷ bubbles/ml. The stability of the suspensions is reported to be better than that of EP-A-0 077 752.

Although the prior art compositions have merit, they still suffer several drawbacks which hamper their practical use. Firstly, some prior art compositions have relatively short life spans and secondly, they have a relatively low initial bubble count e.g. between 10⁴ and 10⁵ bubbles/ml. This makes reproducibility and analysis of echographic tests made with such compositions fairly difficult. In addition, some techniques produce bubbles in a wide range of diameters (up to 50 μm) which prevents their use as echographic agents in certain applications (e.g. echography of the left heart).

The need for stable formulations of microbubbles which will resist pressure variations in the blood streams and have a good shelf life is further amplified by poor stability of some of the state-of-the-art compositions. Microbubble formulations whose distribution and storage would not present problems are particularly important. More specifically, the prior art indicates a need for contrast agent formulations which may be stored as a dry powder for considerable periods of time and at a broad range of temperatures and yet be reliably reconstituted to yield a stable microbubble suspension whose echogenicity has not been compromised by the storage time or temperature.

Another drawback is that many of the heretofore known compositions contain a high amount of different solutes such as polymers, phospholipids, electrolytes, and other which render their practical use more and more difficult. For example, it is known that use of polyoxyethylene/polyoxypropylene polymers (Pluronic®) with particular patients may cause unpleasant side effects (see for instance G. M. Vercellotti et. al. Blood (1982) 59, 1299). Preparations with a high phospholipid content in certain cases may also be undesirable. In any event, compositions with a high degree of various solutes are administered reluctantly and their wide spread use is becoming considered to be undesirable. In fact, the trend in the pharmaceutical industry is to reduce concentrations of active and inactive ingredients in various medical or pharmaceutical formulations to their lowest possible levels and eliminate from the preparations everything that is not necessary. Finding alternative methods and formulating more effective compositions continues to be important. This is particularly so with microbubble suspensions used in echography since here the ingredients have no curative effect and should lead to the least possible after consequences. However, as stated above, the state of the art preparations with typical concentrations in the range of 1% and 4% by weight and the teachings of prior art discourage use of reduced amounts of phospholipids and other non-phospholipid additives. The reason for the discouragement is most probably hidden in the fact that in the course of the routine experimentation further reduction in concentration of the ingredients never produced suspensions which were stable enough to have any practical use or encourage further tinkering in the lower end of the known range.

### SUMMARY OF THE INVENTION

The present invention is based on the unexpected finding that very stable suspensions of gas filled microbubbles comprising at least 10⁷ microbubbles per milliliter may be obtained using phospholipids as stabilizers even if very low concentrations thereof are employed. The suspensions usable as contrasting agents in ultrasonic echography are obtained by suspending in an aqueous carrier at least one phospholipid as a stabiliser of the microbubbles against collapse with time and pressure, the concentration of the phospholipids being below 0.01% wt. but equal to or higher than that at which the phospholipid molecules are present solely at the gas microbubble-liquid interface.

It was quite unexpected to discover that as negligible amounts of the phospholipid surfactants involved here (used alone or with a relatively small proportions of other amphiphiles) can so effectively stabilize microbubbles. In the presence of other amphipathic compounds (such as Pluronic®) the mutual cohesion between stabilizer molecules is apparently decreased and formation of monomolecular phospholipid films is inhibited. However, in the absence of large amounts of other amphiphilic agents, the unhindered intermolecular binding forces (electrostatic interaction or hydrogen bonding) between phospholipid molecules are sufficient to ensure formation of stable film-like structures stabilizing the bubbles against collapse or coalescence.

According to the invention, suspensions of high microbubble concentration, high stability, long storage capacity and ease of preparation may be obtained even if the concentrations of surfactants and other additives in the suspensions are kept well below the levels used in the state-of-the-art formulations. The amount of phospholipids used in the compositions of the invention may be as low as about that only necessary for formation of a single monolayer of the surfactant around the gas microbubbles while the concentration of the bubbles in the suspension is maintained above 10⁷ microbubbles per millilitre. In the present invention, microbubbles with a liposome-like double layer of surfactant (gas filled liposomes) are not likely to exist and have not been observed. Instead, as discussed in more detail infra, the microbubbles are bounded by a mono-molecular layer of surfactant molecules.

The invention further includes dry formulations which may be used to generate the injectable suspensions of the invention by simply mixing with an aqueous carrier phase. These dry formulations are stable when stored over time and at temperatures above ambient temperature. Indeed, the preferred dry formulations of the invention may be reconstituted to generate injectable suspensions of gas filled microbubbles whose echogenicity is unaffected even after storage for a month at 40° C.

### DETAILED DESCRIPTION OF THE INVENTION

Suspensions with high microbubble concentrations e.g. between 10⁹ and 10¹⁰ bubbles/ml of relatively high stability and long storage capacity may be prepared even if the concentration of the phospholipid surfactants are kept well below the levels known in the art. Suspensions with as little as 1 μg of phospholipids per ml may be prepared as long as the amount of the surfactants used is not below that which is necessary for formation of a single monolayer of the lipids around the gas microbubbles and as long as they are produced according to one of the methods herein disclosed.

Calculations have shown that for bubble concentrations of 10⁸ bubbles/ml depending on the size distribution of the microbubbles this concentration may be as low as 1 μg/ml or 0.0001%, however, the phospholipid concentrations between 0.0002% and up to 0.01% are preferred. More preferably the concentration of the phospholipids in the stable suspensions of microbubbles of the invention is between 0.001% and 0.009%. Although further reduction of the amount of phospholipids in the suspension is possible, suspensions prepared with less than 0.0001% wt. are unstable, their total bubble count is low and their echographic response upon injection is not satisfactory. On the other hand, suspensions prepared with more than 0.01% of phospholipids upon injection do not perform better i.e. their stability and echographic response do not further improve with the concentration. Thus, the higher concentrations may only increase the probability of undesirable side effects as set out in the discussion of the prior art. It is tentatively postulated that only the segments of the surfactants which are in the lamellar or laminar form can effectively release molecules organized properly to stabilize the bubbles. This may explain why the concentration of the surfactant may be so low without impairing the stability of the gas bubbles.

The suspensions of the invention offer important advantages over the compositions of the prior art not only because of the low phospholipid content but also because the total amount of injected solutes i.e. lipids and/or synthetic polymers and other additives is between 1,000 and 50,000 times lower than heretofore. This is achieved without any loss of microbubble concentration i.e. echogenicity or stability of the product. In addition to the very low concentration of solutes, the invention provides suspensions which may contain only the microbubbles whose contribution to the echographic signal is relatively significant i.e. suspensions which are free of any microbubbles which do not actively participate in the imaging process.

Needless to say that with such low concentrations of solutes in the injectable composition of the invention probability of undesirable side effects is greatly reduced and elimination of the injected agent is significantly improved.

The microbubble suspensions with low phospholipid content of the invention may be prepared from the film forming phospholipids whose structure has been modified in a convenient manner e.g. by freeze-drying or spray-drying solutions of the crude phospholipids in a suitable solvent. Prior to formation of the suspension by dispersion in an aqueous carrier the freeze dried or spray dried phospholipid powders are contacted with air or preferably, another gas discussed herein, such as a fluorinated gas. When contacted with the aqueous carrier the powdered phospholipids whose structure has been disrupted will form lamellarized or laminarized segments which will stabilise the microbubbles of the gas dispersed therein. Conveniently, the suspensions with low phospholipid content of the invention may also be prepared with phospholipids which were lamellarized or laminarized prior to their contacting with air or another gas. Hence, contacting the phospholipids with air or another gas may be carried out when the phospholipids are in a dry powder form or in the form of a dispersion of laminarized phospholipids in the aqueous carrier.

The term lamellar or laminar form indicates that the surfactants are in the form of thin films or sheets involving one or more molecular layers. As described in WO-A-91/15244 conversion of film forming surfactants into lamellar form can easily be done by, for example, any liposome forming method, for instance by high pressure homogenisation or by sonication under acoustical or ultrasonic frequencies. The conversion into lamellar form may also be performed by coating microparticles (10 μm or less) of a hydrosoluble carrier solid (NaCl, sucrose, lactose or other carbohydrates) with a phospholipid with subsequent dissolution of the coated carrier in an aqueous phase. Similarly, insoluble particles, e.g. glass or resin microbeads may be coated by moistening in a solution of a phospholipid in an organic solvent following by evaporation of the solvent. The lipid coated microbeads are thereafter contacted with an aqueous carrier phase, whereby liposomic vesicles will form in the carrier phase. Also, phospholipids can be lamellarized by heating slightly above critical temperature (Tc) and gentle stirring. The critical temperature is the temperature of gel-to-liquid transition of the phospholipids.

Practically, to produce the low phospholipid content suspensions of microbubbles according to the invention, one may start with liposome suspensions or solutions prepared by any known technique as long as the liposomic vesicles are “unloaded”, i.e. they do not have encapsulated therein any foreign material but the aqueous phase of the solution itself.

The introduction of gas into a liposome solution can be effected by usual means, injection i.e. forcing gas through tiny orifices into the liposome solution, or simply dissolving the gas in the solution by applying pressure and then suddenly releasing the pressure. Another way is to agitate or sonicate the liposome solution in the presence of physiologically acceptable gas. Also one can generate the formation of a gas within the solution of liposomes itself, for instance by a gas releasing chemical reaction, e.g. decomposing a dissolved carbonate or bicarbonate by acid.

When laminarized surfactants are suspended in an aqueous liquid carrier and gas is introduced to provide microbubbles, the microbubbles become progressively surrounded and stabilised by a monomolecular layer of surfactant molecules and not a bilayer as in the case of liposome vesicles. This structural rearrangement of the surfactant molecules can be activated mechanically (agitation) or thermally. The required energy is lower in the presence of cohesion releasing agents, such as Pluronic®. On the other hand, presence of the cohesion releasing agents in the microbubble formulations reduces the natural affinity between phospholipid molecules having as a direct consequence a reduced stability of the microbubbles to external pressures (e.g. above 20-30 Torr).

As already mentioned, to prepare the low phospholipid content suspensions of the invention, in place of phospholipid solutions, one may start with dry phospholipids which may or may not be lamellarized. When lamellarized, such phospholipids can be obtained for instance by dehydrating liposomes, i.e. liposomes which have been prepared normally by means of conventional techniques in the form of aqueous solutions and thereafter dehydrated by usual means. One of the methods for dehydrating liposomes is freeze-drying (lyophilization), i.e. the liposome solution, preferably containing hydrophilic compounds, is frozen and dried by evaporation (sublimation) under reduced pressure.

In another approach, non-lamellarized or non-laminarized phospholipids may be obtained by dissolving the phospholipid in an organic solvent and drying the solution without going through liposome formation. In other words, this can be done by dissolving the phospholipids in a suitable organic solvent together with a hydrophilic stabiliser substance e.g. a polymer like PVP, PVA, PEG (preferably the PEG polymer has a molecular weight from about 1000 to about 7500, with a molecular weight from about 2000 to about 5000 being preferrred and PEG 4000 being most preferred), etc. or a compound soluble both in the organic solvent and water and freeze-drying or spray-drying the solution. Further examples of the hydrophilic stabiliser compounds soluble in water and the organic solvent are malic acid, glycolic acid, maltol and the like. Any suitable organic solvent may be used as long as its boiling point is sufficiently low and its melting point is sufficiently high to facilitate subsequent drying. Typical organic solvents would be for instance dioxane, cyclohexanol, tertiary butanol, tetrachlorodifluoro ethylene (C₂Cl₄F₂) or 2-methyl-2-butanol however, tertiary butanol, 2-methyl-2-butanol and C₂Cl₄F₂ are preferred. In this variant the criteria used for selection of the hydrophilic stabiliser is its solubility in the organic solvent of choice. The suspensions of microbubbles are produced from such powders using the same steps as with powders of the laminarized phospholipids.

Similarly, prior to effecting the freeze-drying of pre-lamellarized or pre-laminarized phospholipid solutions, a hydrophilic stabiliser compound is dissolved in the solution. However, here the choice of the hydrophilic stabilisers is much greater since a carbohydrate like lactose or sucrose as well as a hydrophilic polymer like dextran, starch, PVP, PVA, PEG and the like may be used.

Hydrophilic stabilizer compounds also aid in homogenising the microbubbles size distribution and enhance stability under storage. Actually making very dilute aqueous solutions (0.0001-0.01% by weight) of freeze-dried phospholipids stabilised with, for instance, a 10:1 to 1000:1 weight ratio of polyethyleneglycol to lipid enables to produce aqueous microbubbles suspensions counting 10⁹-10¹⁰ bubbles/ml (size distribution mainly 0.5-10 Jim) which are stable, without significant observable change, even when stored for prolonged periods. This is obtained by simple dissolution of the dried laminarized phospholipids which have been stored under gas without shaking or any violent agitation. The freeze-drying technique under reduced pressure is very useful because it permits, restoration of the pressure above the dried powders with any of the physiologically acceptable gases dicussed infra, i.e. nitrogen, CO₂, argon, methane, freons (organic compounds containing one or more carbon atoms and fluorine), SF₆, CF₄, etc., whereby after redispersion of the phospholipids processed under such conditions suspensions of microbubbles containing the above gases are obtained. It has been found that the surfactants which are convenient in this invention can be selected from amphipathic compounds capable of forming stable films in the presence of water and gases. The preferred surfactants include the lecithins (phosphatidylcholine) and other phospholipids, inter alia phosphatidic acid (PA), phosphatidylinositol phosphatidylethanolamine (PE), phosphatidyl-serine (PS), phosphatidylglycerol (PG), cardiolipin (CL), sphingomyelins. Examples of suitable phospholipids are natural or synthetic lecithins, such as egg or soya bean lecithin, or saturated synthetic lecithins, such as, dimyristoylphosphatidylcholine, dipalmitoylphosphatidylcholine, distearoylphosphatidylcholine or diarachidoylphosphatidylcholine or unsaturated synthetic lecithins, such as dioleylphosphatidyl choline or dilinoleylphosphatidylcholine, with saturated lecithins being preferred.

Additives like cholesterol and other substances can be added to one or more of the foregoing lipids in proportions ranging from zero to 50% by weight. Such additives may include other non-phospholipid surfactants that can be used in admixture with the film forming surfactants and most of which are known. For instance, compounds like polyoxypropylene glycol and polyoxyethylene glycol as well as various copolymers thereof. Other additives may include the acid form of phospholipids (such as phosphatidylglycerol and phosphatidic acid), dicetylphosphate, fatty acids, ergosterol, phytosterol, sitosterol, lanosterol, tocopherol, propyl gallate, ascorbyl palmitate and butylated hydroxytoluene. The amount of these non-film forming surfactants are usually up to 50% by weight of the total amount of surfactants but preferably between 0 and 30%. Again this means that the concentration of the various additives in the low phospholipid content suspensions of the invention are in the range of 0-0.05% which is more than one hundred times less than in the compositions known so far.

It should also be mentioned that another feature of the suspensions of the invention is a relatively “high” gas entrapping capacity of the microbubbles i.e. high ratio between the amount of the surfactant and the total amount of the entrapped gas. Hence, with suspensions in which the microbubbles have sizes in the 1 to 5 μm range, it is tentatively estimated that the weight ratio of phospholipids present at the gas bubble-liquid interface to the volume of entrapped gas under standard conditions is between 0.1 mg/ml and 100 mg/ml.

In practice all injectable compositions should also be as far as possible isotonic with blood. Hence, before injection, small amounts of isotonic agents may also be added to the suspensions of the invention. The isotonic agents are physiological solutions commonly used in medicine and they comprise aqueous saline solution (0.9% NaCl), 2,6% glycerol solution, 5% dextrose solution, etc.

The invention further concerns a method of making stable suspensions of microbubbles according to claim 1 usable as contrast agents in ultrasonic echography. Basically, the method comprises adapting the concentration of the phospholipids in the suspension of microbubbles stabilized by said phospholipids to a selected value within the limits set forth in the claims. Usually, one will start with a microbubble suspension containing more phospholipids than the value desired and one will reduce the amount of said phospholipids relatively to the volume of gas entrapped in the microbubble, without substantially reducing the count of echogenerating bubbles. This can be done, for instance, by removing portions of the carrier liquid containing phospholipids not directly involved at the gas/liquid interface and diluting the suspension with more fresh carrier liquid. For doing this, one may create within the suspension region (a) where the echogenerating bubbles will gather and region (b) where said bubbles are strongly diluted. Then the liquid in region (b) can be withdrawn by separation by usual means (decantation, siphoning, etc.) and a comparable volume of fresh carrier liquid is supplied for replenishment to the suspension. This operation can be repeated one or more times, whereby the content in phospholipids not directly involved in stabilizing the bubbles will be progressively reduced.

It is generally not desirable to achieve complete removal of the phospholipid molecules not present at the bubble gas/liquid interface as some unbalance from equilibrium may result, i.e. if the depletion is advanced too far, some surfactant molecules at the gas/liquid interface may be set free with consequent bubble destabilization. Experiments have shown that the concentration of phospholipids in the carrier liquid may be decreased down to within the neighborhood of the lower limit set forth in the claims without significant changes in properties and adverse effects. This means that, actually, the optimal phospholipid concentration (within the given limits) will be rather dictated by the type of application i.e. if relatively high phospholipid concentrations are admissible, the ideal concentration value will be near the upper limit of the range. On the other hand, if depending on the condition of the patient to be diagnosed, the absolute value of phospholipids must be further reduced, this can be done without adverse effects regarding microbubble count and echogenic efficiency.

An embodiment of the method comprises selecting a film forming surfactant and optionally converting it into lamellar form using one of the methods known in the art or disclosed hereinbefore. The surfactant is then contacted with gas and admixed with an aqueous liquid carrier in a closed container whereby a suspension of microbubbles will form. The suspension is allowed to stand for a while and a layer of gas filled microbubbles formed is left to rise to the top of the container. The lower part of the mother liquor is then removed and the supernatant layer of microbubbles washed with an aqueous solution saturated with the gas used in preparation of the microbubbles. This washing can be repeated several times until substantially all unused or free surfactant molecules are removed. Unused or free molecules means all surfactant molecules that do not participate in formation of the stabilising monomolecular layer around the gas microbubbles.

In addition to providing the low phospholipid content suspensions, the washing technique offers an additional advantage in that it allows further purification of the suspensions of the invention, i.e. by removal of all or almost all microbubbles whose contribution to the echographic response of the injected suspension is relatively insignificant. The purification thus provides suspensions comprising only positively selected microbubbles, i.e. the microbubbles which upon injection will participate equally in the reflection of echographic signals. This leads to suspensions containing not only a very low concentration of phospholipids and other additives, but free from any microbubbles which do not actively participate in the imaging process.

In a variant of the method, the surfactant which optionally may be in lamellar form, is admixed with the aqueous liquid carrier prior to contacting with gas.

### BRIEF DESCRIPTION OF DRAWINGS

FIG. 1 is graphical presentation of echographic responses as a function of the microbubble concentration for a freshly prepared suspension according to the invention.

Suspensions and the method of making low phospholipid content suspensions of the invention will be further illustrated by the following examples:

### EXAMPLE 1

Multilamellar vesicles (MLVs) were prepared by dissolving 240 mg of diarachidoylphosphatidylcholine (DAPC, from Avanti Polar Lipids) and 10 mg of dipalmitoyl-phosphatidic acid (DPPA acid form, from Avanti Polar Lipids) in 50 ml of hexane/ethanol (8/2, v/v) then evaporating the solvents to dryness in a round-bottomed flask using a rotary evaporator. The residual lipid film was dried in a vacuum dessicator. After addition of water (5 ml), the suspension was incubated at 90° C. for 30 minutes under agitation. The resulting MLVs were extruded at 85° C. through a 0.8 μm polycarbonate filter (Nuclepore®). 2.6 ml of the resulting MLV preparation were added to 47.4 ml of a 167 mg/ml solution of dextran 10'000 MW (Fluka) in water. The resulting solution was thoroughly mixed, transferred in a 500 ml round-bottom flask, frozen at −45° C. and lyophilised under 0.1 Torr. Complete sublimation of the ice was obtained overnight. Thereafter, air pressure was restored in the evacuated container. Various amounts of the resulting powder were introduced in glass vials (see table) and the vials were closed with rubber stoppers. Vacuum was applied via a needle through the stopper and the air removed from vials. Upon evacuation of air the powder was exposed to sulfur hexafluoride gas, SF₆.

Bubble suspensions were obtained by injecting in each vial 10 ml of a 3% glycerol solution in water (through the stopper) followed by gentle mixing. The resulting microbubble suspensions were counted using a hemacytometer. The mean bubble size (in volume) was 2.2 μm.

| Dry weight | Phospholipid conc. | Concentration |
|---|---|---|
| (mg/ml) | (μg per ml) | (bubbles/ml) |
| 0.5 | 8 | 9.0 × 10⁶ |
| 1 | 16 | 1.3 × 10⁷ |
| 5 | 81 | 7.0 × 10⁷ |
| 10 | 161 | 1.4 × 10⁸ |

Preparations were injected to rabbits (via the jugular vein) as well as minipigs (via the ear vein) at a dose of 1 ml/5 kg. In vivo echographic measurements were performed using an Acuson XP128 ultrasound system (Acuson Corp. USA) and a 7 MHz sector transducer. The animals were anaesthetised and the transducer was positioned and then fixed in place on the left side of the chest providing a view of the right and left ventricles of the heart in the case of rabbit and a longitudinal four-chamber view in the case of the minipig. The preparation containing 0.5 mg/ml dry weight gave slight opacification of the right as well as the left ventricle in rabbits and in minipigs. The opacification, however, was superior with the 1, 5 and 10 mg/ml preparations.

### EXAMPLE 2

Lyophilisates were prepared as described in Example 1 with air (instead of SF₆) in the gas phase. The lyophilisates were then suspended in 0.9% saline (instead of a 3% glycerol solution). Similar bubble concentrations were obtained. However, after injection in the rabbit or the minipig the persistence of the effect was shorter e.g. 10-20 s instead of 120 s. Moreover, in the minipig the opacification of the left ventricle was poor even with the 10 mg/ml preparation.

### EXAMPLE 3

MLV liposomes were prepared as described in Example 1 using 240 mg of DAPC and 10 mg of DPPA (molar ratio 95:5). Two milliliters of this preparation were added to 20 ml of a polyethyleneglycol (PEG 2,000) solution (82.5 mg/ml). After mixing for 10 min at room temperature, the resulting solution was frozen during 5 min at −45° C. and lyophilised during 5 hours at 0.2 mbar. The powder obtained (1.6 g) was transferred into a glass vial equipped with a rubber stopper. The powder was exposed to SF₆ (as described in Example 1) and then dissolved in 20 ml of distilled water. The suspension obtained showed a bubble concentration of 5×10⁹ bubbles per ml with a median diameter in volume of 5.5 μm. This suspension was introduced into a 20 ml syringe, the syringe was closed and left in the horizontal position for 24 hours. A white layer of bubbles could be seen on the top of solution in the syringe. Most of the liquid phase (˜16-18 ml) was evacuated while the syringe was maintained in the horizontal position and an equivalent volume of fresh, SF₆-saturated, water was introduced. The syringe was then shaken for a while in order to homogenize the bubbles in the aqueous phase. A second decantation was performed under the same conditions after 8 hours followed by three further decantations performed in four hour intervals. The final bubble phase (batch P145) was suspended in 3 ml of distilled water. It contained 1.8×10⁹ bubbles per ml with a median diameter in volume of 6.2 μm. An aliquot of this suspension (2 ml) was lyophilised during 6 hours at 0.2 mbar. The resulting powder was dissolved in 0.2 ml of tetrahydrofuran/water (9/1 v/v) and the phospholipids present in this solution were analysed by HPLC using a light scattering detector. This solution contained 0.7 mg DAPC per ml thus corresponding to 3.9 μg of phospholipids per 10⁸ bubbles. A Coulter counter analysis of the actual bubble size distribution in batch P145 gave a total surface of 4,6×10⁷ μm² per 10⁸ bubbles. Assuming that one molecule of DAPC will occupy a surface of 50 Å², one can calculate that 1,3 μg of DAPC per 10⁸ bubbles would be necessary to form a monolayer of phospholipids around each bubble. The suspension P145 was than left at 4° C. and the concentration of gas bubbles measured on a regular basis. After 10 days, the product looked as good as after its preparation and still contained 1-1.2×10⁹ bubbles per ml. The exceptional stability was found very surprising considering the extremely low amount of phospholipids in the suspension.

The experiment described above was repeated on a second batch of microbubbles using a shorter decantation time in order to collect preferably larger bubbles (batch P132). The median diameter in volume obtained was 8.8 μm and the total surface determined with the Coulter counter was 22×10⁸ μm² per 108 bubbles. The calculation showed that 6 μg DAPC for 10⁸ bubbles would be necessary to cover this bubble population with a monolayer of DAPC. The actual amount of DAPC determined by HPLC was 20 μg per 10⁸ bubbles. Taking into account the difficulty of obtaining precise estimates of the total surface of the bubble population, it appears that within the experimental error, the results obtained are consistent with coverage of the microbubbles with one phospholipid layer.

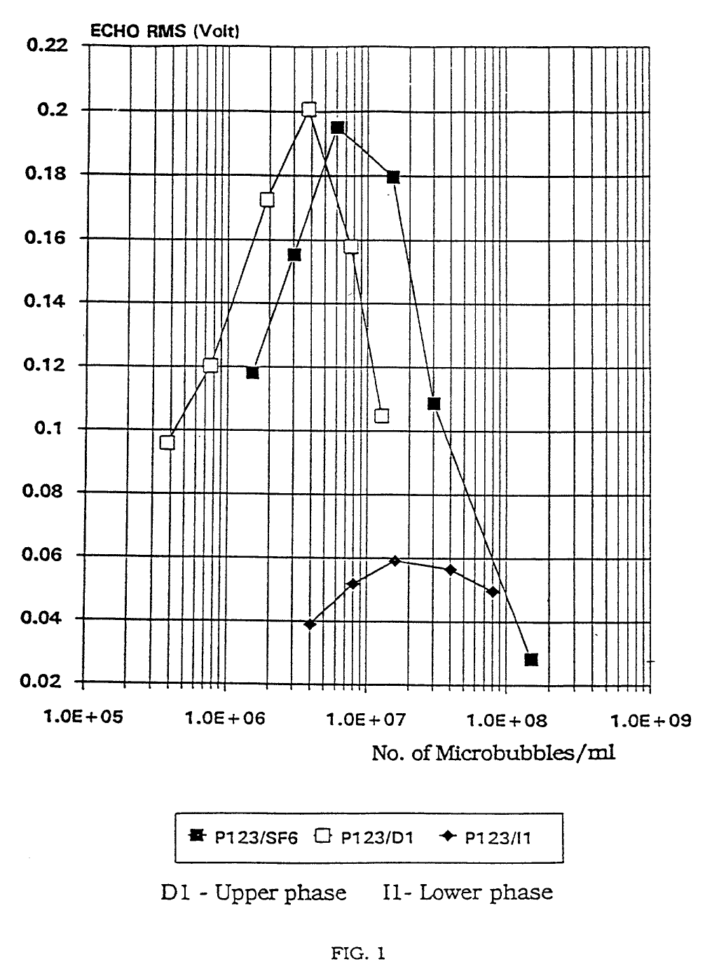

Echographic measurements performed with different washed bubble preparations showed that upon separation the lower phase gives a much weaker echographic signal than the upper phase or a freshly prepared sample. On a first sight this seemed normal as the white layer on the top of the syringe contained the majority of the gas microbubbles anyway. However, as shown in FIG. 1 the bubble count showed a surprisingly high microbubble population in the lower layer too. Only upon Coulter measurement it became apparent that the microbubbles had a size below 0.5 μm, which indicates that with small bubbles even when in high concentration, there is no adequate reflection of the ultrasound signal.

A four fold dilution of the preparation P132 in a 3% glycerol solution was injected in the minipig (0.2 ml/kg). The preparation of washed bubbles containing 2.5×10⁷ bubbles per ml and 5 μg of phospholipids per ml provided excellent opacification in the left and right ventricle with outstanding endocardial border delineation. Good opacification was also obtained by injecting to a minipig an aliquot of preparation P145 (diluted in 3% glycerol) corresponding to 0.2 μg of phospholipids per kg. Contrast was even detectable in the left ventricle after injection of 0.02 μg/kg. Furthermore, in the renal artery the existence of a contrast effect could be detected by pulsed Doppler at phospholipid doses as low as 0.005 μg/kg.

It follows that as long as the laminarized phospholipids are arranged in a single monolayer around the gas microbubbles the suspensions produced will have adequate stability. Thus providing an explanation for the present unexpected finding and demonstrating that the amount of phospholipids does not have to be greater than that required for formation of a monolayer around the microbubbles present in the suspension.

### EXAMPLE 4

A solution containing 48 mg of DAPC and 2 mg of DPPA in hexane/ethanol 8/2 (v/v) was prepared and the solvent evaporated to dryness (as described in Example 1). 5 mg of the resulting powder and 375 mg of polyethyleneglycol were dissolved in 5 g of tert-butanol at 60° C. The clear solution was then rapidly cooled to −45° C. and lyophilised. 80 mg of the lyophilisate was introduced in a glass vial and the powder exposed to SF₆ (see Example 1). A 3% glycerol solution (10 ml) was then introduced in the vial and the lyophilisate dissolved by gentle swirling. The resulting suspension had 1.5×10⁸ bubbles per ml with a median diameter (in volume) of 9.5 μm. This solution was injected to a rabbit providing outstanding views of the right and left ventricle. Even a ten fold dilution of this suspension showed strong contrast enhancement.

### EXAMPLE 5

The procedure of Example 4 was repeated except that the initial dissolution of the phospholipids in hexane/ethanol solution was omitted. In other words, crude phospholipids were dissolved, together with polyethylene glycol in tertiary butanol and the solution was freeze-dried; thereafter, the residue was suspended in water. Several phospholipids and combinations of phospholipids with other lipids were investigated in these experiments. In the results shown in the next table the phospholipids were dissolved in a tertiary butanol solution containing 100 mg/ml of PEG 2'000. The residues obtained after freeze drying were saturated with SF₆ (see Example 1), then dissolved in distilled water at a concentration of 100 mg dry weight per ml.

| | Conc. in tert- | | |
|---|---|---|---|
| Lipid mixture | butanol | Bubble conc. | Median diam. |
| (weight ratio) | (mg/ml) | (× 10⁹/ml) | (μm) |
| DSPC | 2 | 1.3 | 10 |
| DAPC/DPPG (100/4) | 2 | 3.8 | 7 |
| DSPC/Chol (2/1) | 6 | 0.1 | 40 |
| DAPC/Plur F68 (2/1) | 6 | 0.9 | 15 |
| DAPC/Palm. ac. (60/1) | 2 | 0.6 | 11 |
| DAPC/DPPA (100/4) | 1 | 2.6 | 8 |
| DAPC/Chol/DPPA (8/1/1) | 8 | 1.2 | 19 |
| DAPC/DPPA (100/4)* | 5 | 2.4 | 18 |

Legend

ddd

In all cases the suspensions obtained showed high microbubble concentrations indicating that the initial conversion of phospholipids into liposomes was not necessary. These suspensions were diluted in 0.15 M NaCl and injected to minipigs as described in Example 3. In all cases outstanding opacification of the right and left ventricles as well as good delineation of the endocardial border were obtained at doses of 10-50 μg of lipids per kg body weight or less.

### EXAMPLE 6

PEG-2000 (2 g), DAPC (9.6 mg) and DPPA (0.4 mg) were dissolved in 20 ml of tertiary butanol and the solution was freeze dried overnight at 0.2 mbar. The powder obtained was exposed to SF₆ and then dissolved in 20 ml of distilled water. The suspension containing 1.4×10⁹ bubbles per ml (as determined by hemacytometry) was introduced into a 20 ml syringe, which was closed and left in horizontal position for 16 hours. A white layer of bubbles could be seen on top of the solution. The lower phase (16-18 ml) was discarded while maintaining the syringe horizontally. An equivalent volume of fresh SF₆-saturated distilled water was aspirated in the syringe and the bubbles were homogenised in the aqueous phase by agitation. Two different populations of microbubbles i.e. large-sized and medium-sized were obtained by repeated decantations over short periods of time, the large bubbles being collected after only 10-15 min of decantation and the medium sized bubbles being collected after 30-45 min. These decantations were repeated 10 times in order to obtain narrow bubble size distributions for the two types of populations and to eliminate all phospholipids which were not associated with the microbubbles. All phases containing large bubbles were pooled (“large-sized bubbles”). Similarly the fractions containing medium sized bubbles were combined (“medium-sized bubbles”). Aliquots of the two bubble populations were lyophilised and then analysed by HPLC in order to determine the amount of phospholipids present in each fraction. The large-sized bubble fraction contained 2.5×10⁷ bubbles per ml with a median diameter in number of 11.3 μm and 13.7 μg phospholipids per 10⁷ bubbles. This result is in excellent agreement with the theoretical amount, 11.5 μg per 10⁷ bubbles, calculated assuming a monolayer of phospholipids around each bubble and a surface of 50 A per phospholipid molecule. The medium-sized bubble fraction contained 8.8×10⁸ bubbles per ml with a median diameter in number of 3.1 μm and 1.6 μg phospholipids per 10⁷ bubbles. The latter value is again in excellent agreement with the theoretical amount, 1.35 μg per 10⁷ bubbles. These results further indicate that the stability of the microbubble suspensions herein disclosed is most probably due to formation of phospholipid monolayers around the microbubbles.

The following are particular embodiments regarding the suspensions of the invention:

1. An injectable suspension of gas filled microbubbles in an aqueous carrier liquid, usable as contrast agent in ultrasonic echography, comprising at least 10⁷ microbubbles per millilitre and amphipathic compounds at least one of which is a phospholipid stabilizer of the microbubbles against collapse, characterized in that the concentration of the phospholipids in the carrier liquid is below 0.01% by weight while being equal to or above that at which the phospholipid molecules are present solely at the gas microbubble-liquid interface.

2. The injectable suspension of embodiment 1, in which the concentration of microbubbles per millilitre is between 10⁸ and 10¹⁰.

3. The injectable suspension of embodiment 1, in which the concentration of phospholipids is above 0.00013% wt.

4. The injectable suspension of any preceding embodiment, in which the liquid carrier further comprises water soluble poly- and oligo-saccharides, sugars and hydrophilic polymers such as polyethylene glycols as stabilizers.

5. The injectable suspension of any preceding embodiment, in which the phospholipids are at least partially in lamellar or laminar form and are selected from lecithins such as phosphatidic acid, phosphatidylcholine, phosphatidylethanolamine, phosphatidyl-serine, phosphatidylglycerol phosphatidylinositol, cardiolipin and sphingomyelin.

6. The injectable suspension of embodiment 4 or 5, further containing substances affecting the properties of phospholipids selected from phosphatidylglycerol, phosphatidic acid, dicetylphosphate, cholesterol, ergosterol, phytosterol, sitosterol, lanosterol, tocopherol, propylgallate, ascorbyl palmitate and butylated hydroxy-toluene.

7. The injectable suspension of embodiment 1, 2 or 3, in which the phospholipids are in the form of powders obtained by freeze-drying or spray-drying.

8. The injectable suspension of embodiment 1, containing about 10⁸-10⁹ microbubbles per millilitre with the microbubble size between 0.5-10 μm showing little or no variation under storage.

9. The injectable suspension of embodiment 1, in which the liquid carrier further comprises up to 50% by weight non-laminar surfactants selected from fatty acids, esters and ethers of fatty acids and alcohols with polyols such as polyalkylene glycols, polyalkylenated sugars and other carbohydrates, and polyalkylenated glycerol.

10. The injectable suspension of any preceding embodiment, in which the microbubbles are filled with SF₆, CF₄, or freons.

11. A method of making suspensions of gas filled microbubbles comprising selecting at least one film forming surfactant, converting the surfactant into a powder, contacting the powder with gas and admixing the powder surfactant with an aqueous liquid carrier to form said suspension, characterised by introducing the suspension into a container, forming a layer of the gas filled microbubbles in the upper part of the container, separating the layer of the microbubbles formed, and washing the microbubbles with an aqueous solution saturated with the microbubble gas.

12. The method of embodiment 11, in which prior to converting into the powder, the film forming surfactant is at least partially lamellarized.

13. The method of embodiment 12, in which prior to contacting with r gas the partially lamellarized surfactant is admixed with the aqueous liquid carrier

14. The method of embodiment 12 or 13, in which the liquid carrier further contains stabiliser compounds selected from hydrosoluble proteins, polypeptides, sugars, poly- and oligo-saccharides and hydrophilic polymers.

15. The method of embodiment 12, in which the conversion is effected by coating the surfactant onto particles of soluble or insoluble materials leaving the coated particles for a while under gas, and admixing the coated particles with an aqueous liquid carrier.

16. The method of embodiment 12, in which the conversion is effected by sonicating or homogenising under high pressure an aqueous solution of film forming lipids, this operation leading, at least partly, to the formation of liposomes.

17. The method of embodiment 16, in which prior to contacting of at least partially lamellarized surfactant with gas the liposome containing solution is freeze-dried.

18. The method of embodiments 16 and 17, in which the water solution of film forming lipids also contains viscosity enhancers or stabilisers selected from hydrophilic polymers and carbohydrates in weight ratio relative to the lipids comprised between 10:1 and 1000:1.

19. A method of preparation of a suspension of gas filled microbubbles comprising a film forming surfactant, a hydrophilic stabiliser and an aqueous liquid carrier, characterised by dissolving the film forming surfactant and the hydrophilic stabiliser in an organic solvent, freeze drying the solution to form a dry powder, contacting the powder with gas and admixing said powder with the aqueous carrier.

20. The method of embodiment 19, in which the hydrophilic stabiliser is polyethylene glycol, polyvinyl pyrrolidone, polyvinyl alcohol, glycolic acid, malic acid or maltol.

21. The method of embodiment 19 or 20, in which the organic solvent is tertiary butanol, 2-methyl-2-butanol or C₂Cl₄F₂.

22. A method of making an injectable suspension of gas-filled microbubbles according to embodiment 1, which comprises suspending laminarized phospholipids, and optionally other additives, in an aqueous carrier liquid, said phospholipids having been in contact with said gas prior or after being suspended, under conditions such that a concentration of said microbubbles sufficient to provide an echographic respose is formed in the suspension, allowing a portion of said phospholipids to form a stabilization layer around said bubbles and thereafter depleting the carrier liquid of the excess of phospholipids not involved in microbubble stabilization.

As discussed above, while the microbubble suspensions of the invention may employ virtually any biocompatible and amphipathic compound capable of forming stable films in the presence of an aqueous phase and a gas, phospholipids are preferred. Phospholipids useful in the invention include: phosphatidylcholine (PC) with both saturated and unsaturated lipids; including phosphatidylcholine such as dioleylphosphatidylcholine; dimyristoylphosphatidylcholine (DMPC), dipentadecanoylphosphatidylcholine, dilauroylphosphatidylcholine (DLPC); dipalmitoylphosphatidylcholine (DPPC); disteraoylphosphatidylcholine (DSPC); and diarachidonylphosphatidylcholine (DAPC); phosphatidylethanolamines (PE), such as dioleylphosphatidylethanolamine, dipalmitoylphosphatidylethanolamine (DPPE) and distearoylphosphatidylethanolamine (DSPE); phosphatidylserine (PS) such as dipalmitoyl phosphatidylserine (DPPS), disteraoylphosphatidylserine (DSPS); phosphatidylglycerols (PG), such as dipalmitoylphosphatidylglycerol (DPPG), distearoylphosphatidylglycerol (DSPG); and phosphatidylinositol. Saturated phospholipids are particularly preferred. Indeed, in a preferred embodiment disteraoylphosphatidylcholine (DSPC) and dipalmitoylphosphatidylglycerol (DPPG) are used.

As noted above, any physiologically acceptable gas may be present in the agents of the present invention. The term “gas” as used herein includes any substances (including mixtures) substantially in gaseous form at the normal human body (37°). The gas may comprise, for example, air; nitrogen; oxygen; CO₂; hydrogen, nitrous oxide; noble or inert gases such as helium, argon, xenon or krypton; fluorinated gases; and mixtures thereof, with fluorinated gases being preferred. Fluorinated gases include materials which contain at least one fluorine atom such as SF₆, freons (organic compounds containing one or more carbon atoms and fluorine, i.e. CF₄, C₂F₆, C₃F₈, C₄F₈, C₄F₁₀, CBrF₃, CCl₂F₂, C₂ClF₅ and CBrClF₂) and perfluorocarbons. The term perfluorocarbon refers to compounds containing only carbon and fluorine atoms and includes saturated, unsaturated, and cyclic perfluorocarbons such as perfluoroalkanes such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes and perfluoroheptanes; perfluoroalkenes such as perfluoropropene, perfluorobutenes (e.g. perfluorobut-2ene) and perfluorobutadiene; perfluoroalkynes such as perfluorobut-2-yne; and perfluorocycloalkanes such as perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane and perfluorocycloheptane.). The saturated perfluorocarbons, which are usually preferred, have the formula CₙFₙ₊₂, where n is from 1 to 12, preferably from 2 to 10, most preferably from 3 to 8 and even more preferably from 3 to 6. Suitable perfluorocarbons include, for example, CF₄, C₂F₆, C₃F₈ C₄F₈, C₄F₁₀, C₅F₁₂, C₆F₁₂, C₇F₁₄, C₈F₁₈, and C₉F₂₀ In particularly preferred embodiments, SF₆ or perfluorocarbon freons selected from the group consisting of CF₄, C₂F₆ C₃F₈ C₄F₈, and C₄F₁₀are employed in the gas or gas mixture, with use of SF₆, C₃F₈ or C₄F₁₀ being particularly preferred. In a preferred embodiment the microbubbles of the invention contain SF₆.

As cited above the gas can be a mixture of the gases disclosed herein. In particular the following combinations are particularly preferred: a mixture of gases (A) and (B) in which, at least one of the gases (B), present in an amount of between 0.5-41% by vol., has a molecular weight greater than 80 daltons and (B) is selected from the group consisting of SF₆, CF₄, C₂F₆, C₂F₈, C₃F₆, C₃F₅, C₄F₆, C₄F₈, C₄F₁₀, C₅F₁₀, C₅F₁₂ and mixtures thereof and (A) is selected from the group consisting of air, oxygen, nitrogen, carbon dioxide and mixtures thereof, the balance of the mixture being gas A.

In certain circumstances it may be desirable to include a precursor to a gaseous substance (e.g. a material that is capable of being converted to a gas in vivo). Preferably the gaseous precursor and the gas it produces are physiologically acceptable. The gaseous precursor may be pH-activated, photo-activated, temperature activated, etc. For example, certain perfluorocarbons may be used as temperature activated gaseous precursors. These perfluorocarbons, such as perfluoropentane, have a liquid/gas phase transition temperature above room temperature (or the temperature at which the agents are produced and/or stored) but below body temperature; thus, they undergo a phase shift and are converted to a gas within the human body.

As discussed above, the present invention also includes dry formulations which may be used to generate the injectable suspensions of the invention upon simple mixing with an aqueous carrier phase. The dry formulations will generally be in powder or in a cake form and are readily reconstitutable in a suitable aqueous liquid carrier, which is physiologically acceptable, sterile and injectable. Suitable liquid carriers are water, aqueous solutions such as saline (which may advantageously be balanced so that the final product for injection is not hypotonic), or solutions of one or more tonicity adjusting substances such as salts or sugars, sugar alcohols, glycols and other non-ionic polyol materials (eg. glucose, sucrose, sorbitol, mannitol, glycerol, polyethylene glycols, propylene glycols and the like). Reconstitution will generally require only minimal agitation such as may, for example, be provided by gentle hand-shaking. The size of the microbubbles so generated is consistently reproducible and in practice is independent of the amount of agitational energy applied.

The dry formulations will include one or more of the film forming surfactants discussed herein and may include one or more hydrophilic stabilizers and/or addditives. As discussed above, such hydrophilic stabilizers may include a polymer like PVP, PVA, PEG, etc. or a compound soluble both in the organic solvent and water and freeze-drying or spray-drying the solution. Further examples of the hydrophilic stabiliser compounds soluble in water and the organic solvent are malic acid, glycolic acid, maltol and the like. In a preferred embodiment, the hydrophilic stabilizer is polyethylene glycol (PEG) with a molecular weight weight from about 1000 to about 7500, with a molecular weight from about 2000 to about 5000 being preferrred and PEG 4000 being most preferred. The additives may include compounds discussed herein and in certain cases the additives may act as opsonisation inhibitors delaying the uptake of the microbubbles from the vasculature by the reticuloendothelial system.

In practice injectable compositions prepared from the dry formulations should be as close to isotonic with blood as possible. Hence, before injection, small amounts of isotonic agents may also be added to the suspensions of the invention. The isotonic agents are physiological solutions commonly used in medicine and they comprise aqueous saline solution (0.9% NaCl), 2.6% glycerol solution, 5% dextrose solution, etc. Other excipients may optionally be present in the composition being dried or may be added on formulation for administration. Such excipients may for example include pH regulators, osmolality adjusters, viscosity enhancers, emulsifiers, bulking agents, etc. and may be used in conventional amounts.

The preferred dry formulations of ultrasound contrast agents of the present invention not only provide advantages for transport and storage due to the reduction in bulk relative to aqueous dispersions, but they also provide other advantages over freeze-dried products disclosed in the prior art. Specifically, freeze dried products of the prior art are not thermally stable in the range of ambient temperatures normally encountered during transportation and storage and as a result must be maintained in an environment in which the temperature is maintained at or below below ambient (eg. at 5 to 25° C.).

In contrast, the preferred dry formulations of the instant invention are thermally stable at all temperatures normally encountered during transportation and storage. Therefore, these dry formulations may be stored and transported without need of temperature control of the environment and in particular may be supplied to hospitals and physicians for on site formulation into an administrable dispersion without requiring such users to have special storage facilities. Lyophilized products according to the invention have proved to be storage stable for several months under ambient conditions. The microbubble dispersions generated upon reconstitution with an aqueous carrier liquid are stable for considerable lengths of time, eg. up to at least 12 hours, permitting considerable flexibility as to when the dried product is reconstituted prior to injection.

These preferred dry formulations include an additive comprising one or more lipid compounds, which serve as a preserving agent, preventing significant alteration of the acoustic properties of the reconstituted aqueous suspension after storage of the dry formulation over time and at temperatures far exceeding ambient temperature.

This preserving agent is selected from fatty acids, phospholipids in acid form to or a mixture thereof. Additionally, other lipid acids may be used as preserving agents, preferably those having a temperature of fusion greater than 40° C. (Tf>40° C.). While both saturated and unstaurated fatty acids may be used, the preserving agent is preferably a C12-C24 straight chain saturated fatty acid selected from lauric acid, myristic acid, palmitic acid, stearic acid, arachidic acid, behenic acid, lignoceric acid or a mixture thereof. More preferably, the preserving agent is a C14-C20 straight chain saturated fatty acid selected from myristic acid, palmitic acid, stearic acid, arachidic acid or a mixture thereof. In a particularly preferred mode of the present invention, the preserving agent is palmitic acid.

When the preserving agent is chosen among the family of phospholipids in acid form, a saturated phospholipid in acid form selected from dimyristoylphosphatidic acid, dimyristoylphosphatidylglycerol, dimyristoylphosphatidylserine, dipalmitoylphosphatidic acid (DPPA), dipalmitoylphosphatidylglycerol (DPPG), dipalmitoylphosphatidylserine (DPPS), distearoylphosphatidic acid (DSPA), distearoylphosphatidylglycerol (DSPG), distearoylphosphatidylserine (or mixtures thereof) is preferred. Lyso forms of such charged phospholipids are also useful in accordance with the invention, the term “lyso” denoting phospholipids containing only one fatty acyl group. Such lyso forms of phospholipids may advantageously be used in acid form in admixture with phospholipids. One may obtain the acid form of the lipid by protonating it.

The preferred dry formulations of the invention are thermally stable at temperatures in excess of at least 20° C., preferably at least 22° C., 25° C. or 30° C. and especially preferably they are stable at at least 40° C., eg. up to 50° C. Thus, the dry formulation may be stored at a temperature of 20° C., 30° C. or even at 40° C. for a period of one or even two months or more and retain its acoustical properties upon reconstitution. The lipid preserving agents of the present invention prevent the alteration of the acoustic properties (echogenic response) after the reconstitution in an aqueous suspension of a dry powder stored for a period of at least one-month at 40° C.

The preserving agent is present in the dry formulation at a concentration comprised between 1 and 50% by weight of the total amount of the phospholipid film forming surfactant, preferably between 5 and 25% by weight and even more preferably between 10 and 15% by weight of the phospholipid film forming surfactant. In a particularly preferred mode of the present invention, the preserving agent is palmitic acid at a concentration comprised between 10 and 15% by weight of the phospholipid film forming surfactant. As discussed above, after dispersion or reconstitution of the dry formulation in water or in an aqueous carrier liquid, the phospholipid film forming surfactants are present at a concentration in the carrier liquid.below 0.01% by weight.

The lipid preserving agent according to the present invention is a constituent of the membrane and will incorporate into the mono-molecular layer (e.g. the monolayer) surrounding the bubble after reconstitution of the contrast agent in the liquid carrier.

The thermally stable dry formulations of the present invention may be prepared by selecting at least one film forming phospholipid surfactant, converting said phospholipid into a powder, and admixing the powdered phospholipid with one or more lipid preserving agents. The film forming surfactant mixture may be converted to a dry powder by, for example, dissolving the film forming surfactant (with or without the preserving agent) in an organic solvent and freeze drying or spray drying the solution to form a dry powder. Alternatively, the lipid preserving agent can be added to the film forming surfactant mixture prior to its conversion to a powder. Then the surfactant mixture including the preserving agent is converted into a powder.

Prior to reconstitution and optionally before or during packaging, the dry powder is contacted with the desired gas.

When contacted with the aqueous carrier the powdered phospholipids whose structure has been disrupted will form lamellarized or laminarized segments which will stabilise the microbubbles of the gas dispersed therein. The term lamellar or lamella or laminar form indicates that the surfactants are in the form of thin films or sheets involving one or more molecular layers. As described in WO-A-91/15244 (incorporated by reference herein in its entirety) conversion of film forming surfactants into lamellar form can easily be done by any liposome forming method for instance by high-pressure homogenisation or by sonication under acoustical or ultrasonic frequencies. In another embodiment, the suspensions of the present invention may also be prepared with phospholipids which were lamellarized or laminarized prior to their contacting with gas. Hence, contacting the phospholipids with gas may be carried out when the phospholipids are in a dry powder form or in the form of a dispersion of laminarized phospholipids in the aqueous carrier.

If the dry formulation has been stored under or otherwise contacted with the desired gas, reconstitution of the aqueous microbubble suspension of the invention is obtained by simple dissolution of the dry formulation containing the preserving agent without any violent agitation. In a preferred embodiment the film forming surfactant mixture containing the preserving agent is freeze-dried under reduced pressure, permitting the restoration of the pressure above the dried powders with one of the preferred physiologically acceptable gases (i.e SF₆, C₄F₁o or a mixture containing one of these gases). The dry formulation may then be stored under this desired gas until reconstitution with an aqueous carrier is desired.

If the dry formulation has not been contacted with the desired gas during storage or otherwise, reconstitution of the dry formulation is obtained by contacting the powder with the desired gas and admixing said powder with the aqueous carrier.

In a preferred embodiment, the film forming phospholipid suractant(s) and a hydrophilic stabilizer are dissolved in an organic solvent along with a fatty acid preserving agent. The solution is frozen and lyophilized and then the air above the lyophilizate is replaced with the desired gas and the vials of dry formulation are sealed. An echogenic suspension of microbubbles is prepared by reconstituting the dry formulation with saline solution or another physiologically acceptable aqueous liquid carrier.

In one embodiment, the dry formulation is contacted with air or another gas and admixed with an aqueous liquid carrier in a closed container whereby a suspension of microbubbles will form. The suspension is allowed to stand for a while and a layer of gas filled microbubbles formed is left to rise to the top of the container. The lower part of the mother liquor is then removed and the supernatant layer of microbubbles washed with an aqueous solution saturated with the gas used in preparation of the microbubbles. This washing can be repeated several times until substantially all unused or free surfactant molecules are removed. Unused or free molecules mean all surfactant molecules that do not participate in formation of the stabilising monomolecular layer around the gas microbubbles.

In a variant of the preceding embodiment, the dry formulation may be admixed with the aqueous liquid carrier prior to contacting with gas.

As discussed above, the volume and concentrations of the reconstitution liquid may desirably be balanced to make the resulting ready-to-use formulations substantially isotonic. Hence the volume and concentration of reconstitution fluid chosen will be dependent on the type and amount of stabilizer (and/or other bulking agents) present in the freeze-dried product.

The reconstituted contrast agents according to the invention also surprisingly enhance the ability of the microbubbles to retain the fluorinated gases and gas precursors commonly used in the ultrasound contrast agents of the invention.

It will be appreciated that kits can be prepared for use in making the microbubble preparations of the present invention. These kits can include a container containing all of the sterile dry components of the present invention and enclosing the preferred gas or gas mixture in one chamber. The sterile aqueous liquid may be contained in a second chamber of the same container. In one embodiment, the container is a conventional septum-sealed vial. In another, it has a means for directing or permitting application of sufficient bubble forming energy into the contents of the container.

Alternatively, a two container kit may be used in which the dry formulation of the invention may be included in one container together with the desired gas and the sterile aqueous carrier liquid may be included in a separate container in such away that it may be added to the first container under sterile conditions.

The invention has been described above with reference to dry formulations and microbubble suspensions for use as ultrasound contrast agents. However it is also applicable to use of such formulations and suspensions as contrast agents for other diagnostic imaging modalities (e.g. MRI, X-ray, SPECT, PET, magnetic imaging etc.).

As discussed above, the invention comprises in one aspect an injectable aqueous suspension of gas filled microbubbles usable as imaging contrast agent in ultrasonic echography comprising a concentration of phospholipids or other film forming surfactants of below about 0.01% by weight and optionally, additives such as a lipid preserving agent and a hydrophilic stabilizer.

Viewed from another aspect, the invention provides a dry formulation of an ultrasound contrast agent comprising one or more film forming surfactants which may be reconstituted in an aqueous carrier to yield an injectable, echogenic suspension of microbubbles containing less than 0.01% surfactants by weight. In a preferred embodiment the dry formulation further comprises an additive which serves as a preserving agent, permitting the dry formulation to be stored over time and at temperatures considerably higher than ambient temperature while preserving the echogenicity of the reconstituted suspensions. In a particularly preferred embodiment the dry formulation further comprises a hydrophilic stabilizer.

Viewed from a further aspect the invention provides methods of making such dry formulations.

Viewed from a still further aspect the invention provides a method of preparation of a reconstituted suspension starting from the dry formulations disclosed above, characterised by dissolving the phospholipid film forming surfactant, preferably with the preserving agent, in an organic solvent, freeze drying or spray drying the solution to form a dry powder, contacting the powder with gas and admixing said powder with the aqueous liquid carrier.

Viewed from a still further aspect the invention provides the use of one or more lipidic acid as preserving agent (having the ability to prevent the alteration or a significant alteration of the acoustic properties of the reconstituted aqueous suspension after the storage of the dry formulation for a period of at least one month at 40° C.) for the manufacture of an injectable aqueous suspension of gas filled microbubbles for use in diagnosis involving diagnostic ultrasound imaging.

Viewed from a yet still further aspect, the invention provides an injectable reconstituted suspension of gas filled microbubbles usable as ultrasound contrast agents comprising a phospholipid film forming surfactant present at a concentration below 0.01% by weight and an aqueous liquid carrier, characterised in containing one or more lipid preserving agent which, prevents the alteration or a significant alteration of the acoustic properties of the reconstituted aqueous suspension after the storage of the suspension in a dry form for a period of at least one month at 40° C. and optionally a hydrophilic stabilizer or other additive.

Viewed from a further aspect the invention provides a method for imaging an object or body or a region of a body, comprising the steps of: introducing into said object or body or body part or body cavity the injectable reconstituted microbubble suspension as defined above; and then imaging at least a portion of said body by ultrasound or another method of diagnostic imaging (e.g. magnetic resonance imaging etc). According to this method, said body is a vertebrate and said suspension is introduced into the vasculature or body cavity of said vertebrate.

The foregoing description will be more fully understood with reference to the following Examples. These Examples, are, however, exemplary of methods of practising the present invention and are not intended to limit the scope of the invention.

### EXAMPLE 7

### Comparative

### Preparation with DPPA-Na

A solution containing 1 g of DPPC (dipalmitoylphosphatidylcholine) and 100 mg of DPPA-Na (both purchased from Lipoid, Switzerland) was prepared with 50 ml of hexane/isopropanol 8/2 (v/v; Fluka, Switzerland). The solvent was evaporated to dryness. 100 mg of the resulting powder and 10 g of Macrogol 4000 (Clarian, Germany) (PEG 4000) were dissolved in 60 g of tert-butanol at 60° C. to obtain a clear solution. The solution was aliquoted into 100 glass vials of 100 ml and rapidly frozen at −45° C. and lyophilized. The resulting lyophilisate was exposed to SF₆ by replacing air and sealed with stopper within the freeze-dryer (Christ®). The vials of the lyophilisate sample were then stored in ovens at 5, 25, 40° C. and 50° C. in order to perform one month-stability test. To evaluate lyophilisate quality (microbubble formation), the lyophilisate sample was reconstituted with 10 ml saline solution (0.9%-NaCl). Bubble echogenicity (backscatter coefficient measured at 7 MHz, see M. Schneider, Echocardiography: A Jrnal. of CV Ultrasound & Allied Tech., Vol. 16 No.7, part2, 1999 p743-746) and bubble concentration (Coulter Counter) of the suspension were determined. The results of the stability test are summarized below:

TABLE 1

| | Backscatter | Bubble conc. | Bubble Volume |
|---|---|---|---|
| Storage | 10⁻²/(sr · cm) | (10⁸/ml) | (μl/ml) |
| After preparation | 2.8 | 2.3 | 3.4 |
| 1 month at 5° C. | 2.9 | 2.4 | 3.5 |
| 1 month at 25° C. | 2.4 | 1.7 | 3.0 |
| 1 month at 40° C. | 1.5 | 1.2 | 2.2 |
| 1 month at 50° C. | 0.9 | 0.8 | 1.7 |

These results clearly indicate that the bubble sample concentration and echogenicity decrease with the storage. The higher the temperature the lower is the quality of the bubble sample.

### EXAMPLE 8

### Addition of DPPA-H to Improve the Lyophilisate Stability

### (1) Preparation of DPPA-H

Two grams of DPPA-Na were disssolved in a mixed solvent containing chloroform (50 ml), methanol (60 ml) and distilled water (50 ml). After 10 minutes agitation (magnetic stirring), 35 ml of HCl solution (0.1N) was added to the solution and the resulting solution was again agitated for 1 hour at room temperature. Then the solution was poured into a conical ampoule to separate the organic (chloroform) and aqueous phases (H2O+methanol). DPPA-H was finally obtained by eliminating the chloroform and residual solvent by evaporation under reduced pressure without heating and finally by lyophilisation.

### (2) Stability of Lyophilisate Prepared with DPPA-H

Example 7 was reproduced except that all DPPA-Na (sodium form) was replaced by DPPA-H (acid form). The turbidity and Coulter Counter analyses were performed on reconstituted lyophilized samples Oust after the preparation and 1 month later). It was very surprising to find that the stability of the lyophilized sample containing DPPA-H was considerably improved as one can note from Table 2.

TABLE 2

| | Backscatter | Bubble conc. | Bubble Volume |
|---|---|---|---|
| Storage | 10⁻²/(sr · cm) | (10⁸/ml) | (μl/ml) |
| After preparation | 3.4 | 2.5 | 3.7 |
| 1 month at 5° C. | 3.6 | 2.6 | 3.8 |
| 1 month at 25° C. | 3.4 | 2.4 | 3.7 |
| 1 month at 40° C. | 3.5 | 2.7 | 3.6 |
| 1 month at 50° C. | 2.7 | 2.1 | 3.3 |

### EXAMPLE 9

### Addition of DPPG-H to the Lyophilized Preparation

DPPC-H was prepared using the protocol described above for DPPA-H. 100 mg of DSPC, 100 mg of DPPG-Na and 9.8 g of Macrogol 4000 were dissolved in 80 g of tert-butanol under reflux (82° C.). Then the resulting clear solution was equitably divided into two parts in glass bottles. In one solution, 10 mg of DPPG-H was added. After complete dissolution, both two solution samples were frozen and lyophilized. 100 mg of each lyophilisate were placed in glass vials and exposed to gas SF₆. The SF₆ containing lyophilisates were stored at different temperatures for 1 month. The results of the stability test showed that the formulation containing DPPG-H was much more stable than the one, which did not contain DPPG-H.

### EXAMPLE 10

### Addition of Palmitic Acid to the Lyophilized Preparation

The procedure of Example 9 was repeated except that DPPG-H was replaced by palmitic acid. The lyophilized samples were stored in ovens set to different temperatures and the stability was evaluated by comparing lyophilisate with or without palmitic acid (Table 3).

TABLE 3

| | Backscatter coefficient at 7 MHz (10⁻²/(sr · cm)) | |
|---|---|---|
| Storage | without palmitic acid | with palmitic acid |
| After preparation | 3.8 | 3.9 |
| 1 month at 5° C. | 3.7 | 3.8 |
| 1 month at 25° C. | 2.9 | 3.7 |
| 1 month at 30° C. | 2.2 | 3.7 |
| 1 month at 35° C. | 1.5 | 3.6 |
| 1 month at 40° C. | 1.4 | 3.7 |
| 1 month at 50° C. | 0.5 | 2.8 |

The results indicate that addition of a tiny quantity of palmitic acid (0.2% by weight of dried lyophilisate) improved considerably the shelf life of the lyophilisate during storage.

### EXAMPLE 11

### Addition of Various Fatty Acids

The procedure of Example 9 was repeated except that DPPG-H was replaced by 0.2% of one of several negatively charged phospholipids (in acid form) or fatty acids (lauric, myristic, palmitic and stearic acids). The lyophilized samples were exposed to SF₆ and C₄F₁₀ gases, then stored 1 month at 40° C. Stability test was performed as before. Table 4 shows the results (backscatter coefficient %).

TABLE 4
% of the Backscatter coefficient after 1 month at 40° C. relative
to the values at t = 0

| Storage | SF6 | C4F10 |
|---|---|---|
| Without fatty acid | 37 | 41 |
| DSPA-H | 101 | 96 |
| DSPG-H | 99 | 103 |
| DPPS-H | 103 | 98 |
| Lauric acid | 98 | 100 |
| Myristic acid | 101 | 99 |
| Palmitic acid | 102 | 98 |
| Stearic acid | 105 | 102 |

These data show that the negatively charged phospholipids in acid form and fatty acids in general can improve the stability of the microbubbles forming lyophilisate during storage.

### EXAMPLE 12

### Influence of the Amount of DPPG-H

The procedure of Example 9 was repeated with different amounts of DPPG-H (from 0 to 25% of the total lipids used for the preparation). The results of absorbance measurements (see the description in U.S. Pat. No. 5,578,292 incorporated by refernce herein in its entirety), obtained from reconstituted SF₆ filled-lyophilisate at t=0 and one month of storage at 40° C., are set forth in Table 5.

TABLE 5
Absorbance measurements (at t = 0 and t1 = 1 month at 40° C.)
(Abs. 700 nm)

| % DPPG-H | t = 0 | t1 | stability % |
|---|---|---|---|
| 0 | 0.24 | 0.13 | 54 |
| 2 | 0.25 | 0.17 | 68 |
| 5 | 0.25 | 0.21 | 84 |
| 10 | 0.27 | 0.28 | 103 |
| 15 | 0.30 | 0.29 | 97 |
| 20 | 0.23 | 0.20 | 87 |
| 25 | 0.24 | 0.19 | 79 |

### EXAMPLE 13

### Influence of the Amount Palmitic Acid

The procedure of Example 10 was performed with different amounts of pamitic acid (from 0 to 25% of the total lipids used for the preparation). The results of Coulter measurements (bubble concentration) obtained from reconstituted SF₆ filled-lyophilisate at t=0 and after one month of storage at 40° C. (t1), are gathered in Table 6.

TABLE 6
Coulter measurements (at t = 0 and t1 = 1 month at 40° C.)
(bubble conc. 10⁸/ml))

| % Palm. acid | t = 0 | t1 | stability % |
|---|---|---|---|
| 0 | 2.5 | 1.2 | 48 |
| 2 | 2.6 | 1.6 | 62 |
| 5 | 2.5 | 1.9 | 76 |
| 10 | 2.6 | 2.5 | 96 |
| 15 | 2.3 | 2.2 | 96 |
| 20 | 1.5 | 1.3 | 87 |
| 25 | 0.6 | 0.5 | 83 |

We claim:

1. An ultrasound contrast agent comprising an aqueous suspension of gas filled microbubbles comprising a saturated phospholipid, a fatty acid, a hydrophilic stabilizer, and SF₆, wherein the amount of the saturated phospholipid in the suspension is less than about 0.01% by weight.

2. The ultrasound contrast agent of claim 1, wherein the fatty acid is present in an amount between 1% and 50% by weight of the amount of the saturated phospholipid.

3. The ultrasound contrast agent of claim 1, wherein the fatty acid is present in an amount between 5% and 25% by weight of the amount of the saturated phospholipid.

4. The ultrasound contrast agent of claim 1, wherein the fatty acid is present in an amount between 10% and 15% by weight of the amount of the saturated phospholipid.

5. The ultrasound contrast agent of claim 1, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

6. The ultrasound contrast agent of claim 1, wherein the fatty acid comprises palmitic acid in an amount between 10% and 15% by weight of the amount of the saturated phospholipid.

7. The ultrasound contrast agent of claim 1, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

8. The ultrasound contrast agent of claim 1, wherein the saturated phospholipid comprises distearoylphosphatidylcholine (DSPC) and dipalmitoylphosphatidylglycerol (DPPG).

9. The ultrasound contrast agent of any one of claims 1, 6 or 8 wherein the hydrophilic stabilizer comprises PEG 4000.

10. The ultrasound contrast agent of claim 1, wherein the saturated phospholipid comprises distearoylphosphatidylcholine (DSPC) and dipalmitoylphosphatidylglycerol (DPPG), the fatty acid comprises palmitic acid in an amount between 10 and 15% by weight of the amount of the saturated phospholipid, and the hydrophilic stabilizer comprises PEG 4000.

11. A method of imaging a region of a body comprising:
(a) administering to the body an aqueous suspension of gas filled microbubbles comprising a saturated phospholipid, a fatty acid, a hydrophilic stabilizer, and SF₆, wherein the amount of the saturated phospholipid in the suspension is less than 0.01% by weight; and
(b) imaging the body.

12. The method of imaging of claim 11, wherein the fatty acid is present in an amount between 1% and 50% by weight of the amount of the saturated phospholipid.

13. The method of imaging of claim 11, wherein the fatty acid is present in an amount between 5% and 25% by weight of the amount of the saturated phospholipid.

14. The method of imaging of claim 11, wherein the fatty acid is present in an amount between 10% and 15% by weight of the amount of the saturated phospholipid.

15. The method of imaging of claim 11, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

16. The method of imaging of claim 11, wherein the fatty acid comprises palmitic acid in an amount between 10% and 15% by weight of the amount of the saturated phospholipid.

17. The method of imaging of claim 11, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

18. The method of imaging of claim 11, wherein the saturated phospholipid comprises distearoylphosphatidylcholine (DSPC) and dipalmitoylphosphatidylglycerol (DPPG).

19. The method of imaging of any one of claims 11, 16 or 18, wherein the hydrophilic stabilizer comprises PEG 4000.

20. The method of imaging of claim 11, wherein the saturated phospholipid comprises distearoylphosphatidylcholine (DSPC) and dipalrnitoylphosphatidylglycerol (DPPG), the fatty acid comprises palmitic acid in an amount between 10% and 15% by weight of the amount of the saturated phospholipid, and the hydrophilic stabilizer comprises PEG 4000.

21. The method of imaging of claim 11, wherein the body is a vertebrate and the suspension is administered to the vasculature or body cavity of the vertebrate.

22. A dry formulation of an ultrasound contrast agent comprising a saturated phospholipid, a fatty acid, and a hydrophilic stabilizer, wherein upon dissolution in an aqueous carrier liquid, the dry formulation will form a suspension of microbubbles comprising SF₆ in which the amount of saturated phospholipid in the suspension is less than about 0.01% by weight.

23. The dry formulation of claim 22, wherein the fatty acid is present in an amount of between 1% and 50% by weight of the amount of the saturated phospholipid.

24. The dry formulation of claim 22, wherein the fatty acid is present in an amount of between 5% and 25% by weight of the amount of the saturated phospholipid.

25. The dry formulation of claim 22, wherein the fatty acid is present in an amount of between 10% and 15% by weight of the amount of the saturated phospholipid.

26. The dry formulation of claim 22, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

27. The dry formulation of claim 22, wherein the fatty acid comprises palmitic acid in an amount of between 10% and 15% by weight of the amount of the saturated phospholipid.

28. The dry formulation of claim 22, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

29. The dry formulation of claim 22, wherein the saturated phospholipid comprises distearoylphosphatidylcholine (DSPC) and dipalmitoylphosphatidylglycerol (DPPG).

30. The dry formulation of any one of claims 22, 27 or 29, wherein the hydrophilic stabilizer comprises PEG 4000.

31. The dry formulation of claim 22, wherein the saturated phospholipid comprises distearoylphosphatidylcholine (DSPC) and dipalmitoylphosphatidylglycerol (DPPG), the fatty acid comprises palmitic acid in an amount of between 10% and 15% by weight of the amount of the saturated phospholipid, and the hydrophilic stabilizer comprises PEG 4000.

32. A method of using the dry formulation of any one of claims 22 to 29 or 31 for the preparation of an ultrasound contrast agent comprising forming a suspension of gas filled microbubbles with the dry formulation.

33. A method of preparing an ultrasound contrast agent comprising reconstituting the dry formulation of any one of claims 22 to 29 or 31 in an aqueous carrier liquid to form a suspension of gas filled microbubbles.

34. A method of imaging a region of a body comprising:
(a) reconstituting the dry formulation of any one of claims 22 to 29 or 31 in an aqueous carrier liquid to form a suspension of gas filled microbubbles;
(b) administering the suspension of gas filled microbubbles to the body; and
(c) imaging the body.

35. A method of preparing a contrast agent comprising an aqueous suspension of gas filled microbubbles comprising SF₆ and saturated phospholipid, wherein the amount of saturated phospholipid in the suspension is less than about 0.01% by weight, the method comprising:
(a) dissolving at least one saturated phospholipid, a fatty acid, and a hydrophilic stabilizer in an organic solvent to form a solution;
(b) freeze drying or spray drying the solution to form a dried powder;
(c) contacting the dried powder with SF₆; and
(d) mixing the freeze dried or spray dried powder with an aqueous carrier phase.

36. A method of preparing a dry formulation of an ultrasound contrast agent, wherein upon dissolution in an aqueous carrier liquid, the dry formulation will form a suspension of microbubbles comprising SF₆ and saturated phospholipid, wherein the amount of the saturated phospholipid in the suspension is less than about 0.01% by weight, the method comprising:
(a) dissolving at least one saturated phospholipid, a fatty acid, and a hydrophilic stabilizer in an organic solvent to form a solution;
(b) freeze drying or spray drying the solution to form a dried powder; and
(c) contacting the dried powder with SF₆.

37. A method of preparing a dry formulation of an ultrasound contrast agent, wherein upon dissolution in an aqueous carrier liquid, the dry formulation will form a suspension of microbubbles comprising SF₆ and saturated phospholipid, wherein the amount of the saturated phospholipid in the suspension is less than about 0.01% by weight, the method comprising:
(a) dissolving at least one saturated phospholipid and a hydrophilic stabilizer in an organic solvent to form a solution;
(b) freeze drying or spray drying the solution to form a dried powder;
(c) mixing the dried powder with a fatty acid to form a mixture; and
(d) contacting the mixture with SF₆.

38. An ultrasound contrast agent comprising an aqueous suspension of gas filled microbubbles comprising a saturated phospholipid, a preserving agent, SF₆, and a hydrophilic stabilizer, wherein the amount of saturated phospholipid in the suspension is less than about 0.01% by weight, and the preserving agent comprises a fatty acid.

39. The ultrasound contrast agent of claim 38, wherein the preserving agent is present in an amount between 1% and 50% by weight of the amount of the saturated phospholipid.

40. The ultrasound contrast agent of claim 38, wherein the preserving agent is present in an amount between 5% and 25% by weight of the amount of the saturated phospholipid.

41. The ultrasound contrast agent of claim 38, wherein the preserving agent is present in an amount between 10% and 15% by weight of the amount of the saturated phospholipid.

42. The ultrasound contrast agent of claim 38, wherein the preserving agent is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

43. The ultrasound contrast agent of claim 38, wherein the preserving agent comprises palmitic acid in an amount between 10% and 15% by weight of the amount of the saturated phospholipid.

44. The ultrasound contrast agent of claim 38, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

45. The ultrasound contrast agent of claim 38, wherein the saturated phospholipid comprises distearoylphosphatidylcholine (DSPC) and dipalmitoylphosphatidylglycerol (DPPG).

46. The ultrasound contrast agent of any one of claims 38, 43 or 45, wherein the hydrophilic stabilizer comprises PEG 4000.

47. The ultrasound contrast agent of claim 38, wherein the saturated phospholipid comprises distearoylphosphatidylcholine (DSPC) and dipalmitoylphosphatidylglycerol (DPPG), the preserving agent comprises palmitic in an amount between 10% and 15% by weight of the amount of the saturated phospholipid, and the hydrophilic stabilizer comprises PEG 4000.

48. A dry formulation of an ultrasound contrast agent comprising a saturated phospholipid, a preserving agent, and a hydrophilic stabilizer, wherein the preserving agent comprises a fatty acid, and upon dissolution in an aqueous carrier liquid, the dry formulation will form a suspension of microbubbles comprising SF₆ in which the amount of saturated phospholipid in the suspension is less than about 0.01% by weight.

49. The dry formulation of claim 48, wherein the preserving agent is present in an amount between 1% and 50% by weight of the amount of the saturated phospholipid.

50. The dry formulation of claim 48, wherein the preserving agent is present in an amount between 5% and 25% by weight of the amount of the saturated phospholipid.

51. The dry formulation of claim 48, wherein the preserving agent is present in an amount between 10% and 15% by weight of the amount of the saturated saturated phospholipid.

52. The dry formulation of claim 48, wherein the preserving agent is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

53. The dry formulation of claim 48, wherein the preserving agent comprises palmitic acid in an amount between 10% and 15% by weight of the amount of the saturated phospholipid.

54. The dry formulation of claim 48, wherein the saturated phospholipid is selected from the group consisting of dimyristoylphosphatidic acid, dimyristoylphosphatidylglycerol, dimyristoylphosphatidylserine, dipalmitoylphosphatidic acid, dipalrnitoylphosphatidylglycerol, dipalmitoylphosphatidylserine, distearoylphosphatidic acid, distearoylphosphatidylglycerol, distearoylphosphatidylserine and mixtures thereof.

55. The dry formulation of claim 48, wherein the saturated phospholipid comprises distearoylphosphatidylcholine (DSPC) and dipalmitoylphosphatidylglycerol (DPPG).

56. The dry formulation of any one of claims 48, 53 or 55, wherein the hydrophilic stabilizer comprises PEG 4000.

57. The dry formulation of claim 48, wherein the saturated phospholipid comprises distearoylphosphatidylcholine (DSPC) and dipalmitoylphosphatidylglycerol (DPPG), the preserving agent comprises palmitic acid in an amount between 10% and 15% by weight of the amount of the saturated phospholipid, and the hydrophilic stabilizer comprises PEG 4000.

58. A method of using the dry formulation of any one of claims 48 to 55 or 57 for the preparation of an ultrasound contrast agent comprising forming a suspension of gas filled microbubbles from the dry formulation.

59. A method of preparing an ultrasound contrast agent comprising reconstituting the dry formulation of any one of claims 48 to 55 or 57 in an aqueous carrier liquid to form a suspension of gas filled microbubbles.

60. A method of imaging a region of a body comprising:
(a) reconstituting the dry formulation of any one of claims 48 to 55 or 57 in an aqueous carrier liquid to form a suspension of gas filled microbubbles;
(b) administering the suspension of gas filled microbubbles to the body; and
(c) imaging the body.
